(19) United States
(12) Patent Application Publication
(10) Pub. No.: US 2011/0023871 A1
(43) Pub. Date: Feb. 3, 2011

(54) SEMI-RIGID STYLET FOR ADVANCEMENT OF DOUBLE LUMEN ENDOTRACHEAL VENTILATING TUBES

(75) Inventor: John Allen Pacey, Vancouver (CA)

Correspondence Address: BLACK LOWE & GRAHAM, PLLC, 701 FIFTH AVENUE, SUITE 4800, SEATTLE, WA, 98104, US

(73) Assignee: Verathon Inc., Bothell, WA (US)

(21) Appl. No.: 12/848,767

(22) Filed: Aug. 2, 2010

Related U.S. Application Data
(60) Provisional application No. 61/230,965, filed on Aug. 3, 2009.

(51) Int. Cl. A61M 16/00

(52) U.S. Cl. 128/200.26

(57) ABSTRACT

Semi-rigid stylets configured with a particular curve shaped and handle design to accurately advance either left-sided or right-sided double lumen endotracheal tubes to be positioned in the appropriate place are described. The semi-rigid stylets have a distal bend to counter or compensate for the inherent curvature of double lumen tubes imparted by the memory of spooled tubing stocks. Other embodiments of the semi-rigid stylets are made of materials that establish only those forces sufficient to cause the necessary bending to route a bronchial cuff to a desired lung location without causing substantial tissue injury. The semi-rigid stylets are also fitted with a curved handle configured to engage a user's thumb for easy manipulation and includes a locking component to engage a nearby tracheal tube extension to stabilize endotracheal tube shapes for insertion into the trachea and bronchial regions.

### CROSS REFERENCES TO RELATED APPLICATIONS

This application incorporates by reference in its entirety and claims the benefit of priority to U.S. Provisional Patent Application No. 61/230,965 filed Aug. 3, 2010.

### FIELD OF THE INVENTION

The present invention is in the field of anesthesiology, and, in particular, apparatuses that provide intubation and examination of internal organs, such as the lungs, in surgical and anesthesiological procedures.

### BACKGROUND OF THE INVENTION

Double lumen endotracheal intubation provides the current preferred method for control of the airway utilizing anesthesiology procedures involving mechanical ventilation of patients undergoing surgery in the thoracic cavity. The thoracic operations target the lungs and other organs in the chest such as the esophagus that must be performed with endotracheal tubes that have more than one lumen. These tubes, commonly called double lumen tubes (DLT), are bulky and stiff so that they resist angulation and manipulation. Oftentimes these tubes require a special purpose stylet that will accommodate the DLT and control its complex passage into the trachea and toward the appropriate bronchial lumen by usual techniques The standard stylets used to place these tubes are too soft to be effective in routing the DLT, particularly when a video laryngoscope such as the Glidescope® is used to permit viewing of the tracheal and/or bronchial passages.

### BRIEF DESCRIPTION OF THE DRAWINGS

Preferred and alternative examples of the present invention are described in detail below with reference to the following drawings:

FIG. 1 schematically depicts features of a semi-rigid stylet 10;

FIG. 2 schematically depicts the semi-rigid stylet 10 adjacent to a double lumen endotracheal tube;

FIG. 3 schematically depicts the semi-rigid stylet 10 positioned in the bronchial channel of a left-sided double lumen endotracheal tube;

FIG. 4 schematically depicts the semi-rigid stylet 10 positioned in and partially retracted from the bronchial channel of a right-sided double lumen endotracheal tube;

FIG. 5 schematically depicts an alternate embodiment of the semi-rigid stylet fitted with an adjustable plug engaged with the endobronchial tube extender.

FIG. 6 is a perspective view that schematically depicts an alternate embodiment of a stylet 200 fitted with a handle grasp;

FIG. 7 depicts a side cross-sectional view of the stylet 200;

FIG. 8 depicts a rear view of the stylet 200;

FIG. 9 depicts a side view of the stylet 200;

FIG. 10 depicts a top view of the stylet 200;

FIG. 11 depicts top view of the stylet handle 222;

FIG. 12 depicts top view of the stylet handle 222 illustrating alphanumeric information formed thereon; and

FIG. 13 schematically depicts the semi-rigid stylet 200 positioned in the bronchial channel of a right-sided double lumen endotracheal tube to position the ball 230 beyond the bronchial lumen orifice 68.

### DETAILED DESCRIPTION OF THE PARTICULAR EMBODIMENTS

An embodiment encompassing semi-rigid stylets configured with a particular curve shaped and handle design to accurately advance either left-sided or right-sided double lumen endotracheal tubes to be positioned in the appropriate place is described below. The semi-rigid stylets have a distal bend to counter or compensate for the inherent curvature of double lumen tubes imparted by the memory of spooled tubing stocks. Other embodiments of the semi-rigid stylets are made of materials that establish only those forces sufficient to cause the necessary bending to route a bronchial cuff to a desired lung location without causing substantial tissue injury. The semi-rigid stylets are also fitted with a curved handle configured to engage a user's thumb for easy manipulation and includes a locking component to engage a nearby tracheal tube extension to stabilize endotracheal tube shapes for insertion into the trachea and bronchial regions.

Double lumen tubes have a first tube extension engaged with one of the lumen channels, and a second tube extension engaged with the other lumen channel of the double lumen tube. In alternate embodiments, the locking component may engage either the first tube extension or the tracheal tube extension and the second tube extension or the bronchial tube extension of the double lumen tube.

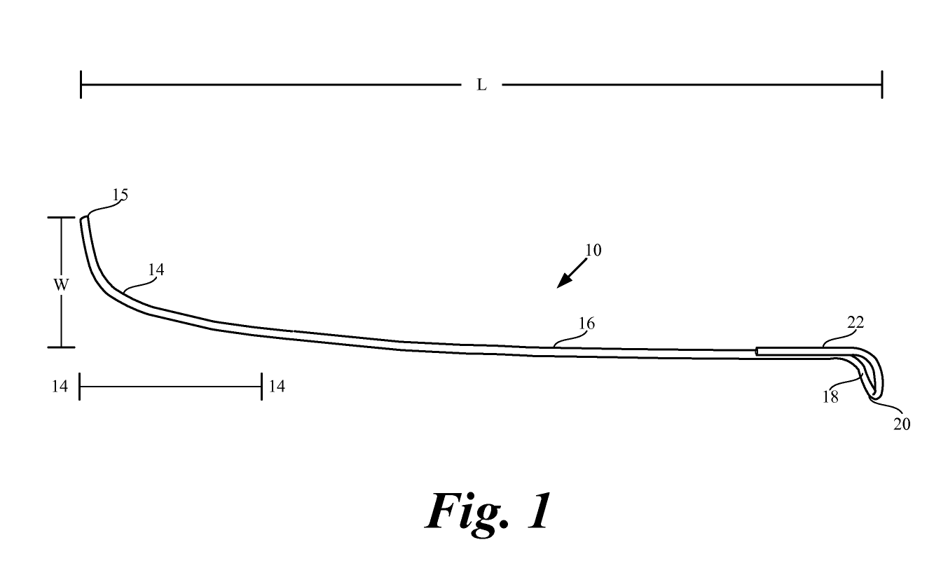

More particularly, the stylet embodiments having new utility features are illustrated in FIGS. 1-5 below. One embodiment includes a stylet having a curve that is in the distal 25-30% of the length that has a radius that can permit entry into the stiff proximal portions of a double lumen tube and the radius is sufficient to change the direction of the stylet's metal shaft from 45-110 degrees but the usual directional change is in the range of 90 degrees. More complete circles can be as much as 180 degrees but in this case the radius would extent to 50% or more of the total length of the stylet.

Another embodiment provides for a stylet having sufficient rigidity which may be only deformed with significant amount of force (forces that are not likely to be produced in the act of intubation in that excessive applied forces would cause the stylet deformation and possibly cause tissue injury as a consequence of too much applied force. The added rigidity reduces the potential for deviation during placement).

Another embodiment of the stylet provides for a return curvature on the proximal end of the stylet which at once allows the thumb to be used for partial extraction of the stylet and a portion of the stylet which passes back toward the tip of the stylet (distal) and engages the second lumen of the endotracheal tube and prevents rotation of the endotracheal tube on the stylet. The return curvature of the stylet provides that the endotracheal tube be mounted in such a way that the natural curvature of the endotracheal tube plastic memory is reversed. The reversal of the curvature results in the bronchial tube portion of the endotracheal tube being diverted from left to right in a left-sided double lumen tube and for the cuff to be kept on the right with a right-sided double lumen tube. Yet another embodiment of the stylet employs a stylet aid in the form of a sliding block that engages the double lumen tube proximally to allow some adjustment for length. The length of some double lumen tubes is longer than others.

FIG. 1 schematically depicts features of a semi-rigid stylet 10. The semi-rigid stylet 10 includes a curved distal section 14 that terminates at stylet tip 15, a substantially linear or gradually curved middle section 16, a proximal handle section 18 having a downwardly disposed thumb grasp 20, and a securing re-curved bar 22 that loops backward from the thumb grasp 20. The length L may be defined as the approximate distance between the stylet tip 15 and the nadir of the thumb grasp 20. The curved distal section 14 approximates a length of 25-30% of length L denoted approximately by bar 14-14. Length L may be approximately 390 millimeters (mm), and in alternate embodiments, may be increased or decreased to accommodate the lengths of the dual lumen tubes used in anesthesiology procedures for pediatric to adult size patients. The distal curve may vary between 98 and 117 mm at 25-30% of 390 mm. The curved width denoted by bar W that approximates the distance from stylet tip 15 and linear section 16 is approximately 67 mm for the stylet 10 having a length of approximately 390 mm. The diameter of the stylet 10 may be approximately 3.2 mm. Material used in the construction of the stylet 10 may be made from ASTM and/or ISO medical grade materials that impart the semi-rigidity and be tolerant to disinfection and/or sterilization processes employed in the health care industry. The stylet 10 may be constructed of grade 316-80 stainless steel having an approximate 3.2 mm diameter.

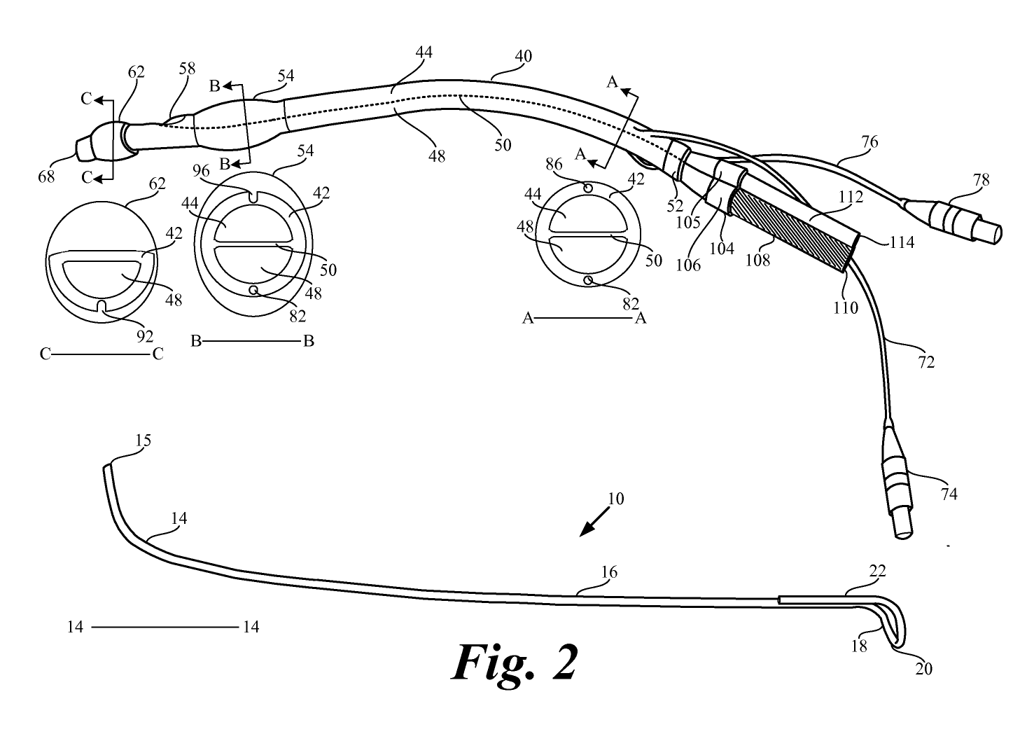

FIG. 2 schematically depicts the semi-rigid stylet 10 adjacent to a left sided double lumen endotracheal tube 40 or DLT 40. The DLT 40 has two parallel channels or lumens comprising a tracheal lumen 44 and a bronchial lumen 48, each being separated by a lumen partition 50. The tracheal lumen 44 may be shorter than the bronchial lumen 48. The DLT 40 has a proximal end 52. A tracheal cuff 54 is shown adjacent to the distal tracheal orifice 58. The tracheal cuff 54 wraps around both the exteriors of the tracheal lumen 44 and bronchial lumen 48. The tracheal lumen 48 extends distally from the tracheal orifice 58 where a bronchial cuff 60 is shown wrapping around the exterior of the bronchial lumen extension. A distal bronchial orifice 68 denotes the more distal part of the DLT 40.

Also shown in FIG. 2 are proximal cross-section line A-A, distal cross-section line B-B, and distal cross-section line C-C, each cross-section line having their respective cross-section views A-A, B-B, and C-C adjacently nearby. The cross-sectional views are magnified to illustrate additional details. Cross-sectional view A-A illustrates the proximal end of the DLT 40, view B-B the tracheal cuff section, and view C-C the bronchial cuff section. Between cross-section line A-A and proximal end 52 are shown air supply lines 72 and 76 that respectively merge into the wall 42 surrounding tracheal lumen 44 and bronchial lumen 48. Air supply line 72 may be connected with syringe port 74 and air supply line 76 may be connected with syringe port 78. As shown in cross-section view A-A, the air supply line 72 merges into bronchial cuff air channel 82 and air supply line 76 merges into tracheal cuff air channel 86. Lumen partition 50 is shown splitting the tracheal lumen 44 and bronchial lumen 48 into approximately evenly sized hemispheres that remain pneumatically isolated from each other due to the intervening presence of lumen partition 50. In alternate embodiments the DLT 40 may include two substantially parallel running tubular sections adhered together, with one tube functioning as the tracheal lumen 44 and the other tubular section functioning as the bronchial lumen 48.

Illustrated at cross-section tracheal cuff view B-B, the tracheal cuff air channel 86 opens into orifice 96 to pneumatically communicate between syringe port 74 and tracheal cuff 54. Syringe manipulation at syringe port 74 can inflate or deflate the tracheal cuff 54. Similarly illustrated at cross-section bronchial cuff view C-C, the bronchial cuff air channel 82 opens into orifice 92 to pneumatically communicate between syringe port 78 and bronchial cuff 62. Syringe manipulation at syringe port 78 can inflate or deflate the bronchial cuff 62.

Attached to the distal end 52 is a double channel tube connector 104 that partitioned to keep air supply and return separated via a tracheal section 105 and a bronchial section 106. Attached to tracheal section 105 is tracheal tube extension 112, and to bronchial section 106 is attached bronchial tube extension 108. The tracheal tube extension 112 includes an opening 114 and the bronchial tube extension 108 includes an opening 110. In alternate embodiments the bronchial tube extension 108 may be colored, as indicated by the lined pattern, and/or with a hue similar to the bronchial cuff 62. The tracheal tube extension 112 may be clear to match the clear appearance of the tracheal cuff 54.

As illustrated in FIG. 2, the left sided double lumen tube 40 has a natural curvature that is the reverse of the stylet 10. That is, the clockwise direction of the stylet 10 when viewing from the proximal handle 18 towards the style tip 15 is offset by the counter-clockwise direction of the DLT 40 when viewed from the proximal end 52 towards the distal bronchial orifice 68. The stylet 10 may be fitted through the bronchial entry port 110 and into the bronchial channel 48 to and through the bronchial exit orifice 68. Similarly, the stylet 10 may also be fitted through the tracheal entry port 114 and into the tracheal channel 44 to and through the tracheal exit orifice 58. Coursing the stylet 10 through either the tracheal channel 44 or the bronchial channel 48 serves to straighten the DLT 40 due to the clockwise stylet 10 configuration counter-balancing or compensating for the counter-clockwise orientation of the DLT 40. Depending on whether the DLT 40 is right-sided or left-sided, and to the extent that the stylet 10 is pushed, pulled, or rotated within the respective bronchial lumen 48 or tracheal lumen 44, a particular directional route of the distal bronchial orifice 68 or distal tracheal orifice 58 may be custom developed as needed when needed for a given patient's lung anatomy. The return curvature on the proximal end of the stylet 10 allows the thumb to be used for partial extraction by engaging with the thumb grasp 20 of the stylet 10 and a portion of the stylet which passes back toward the tip of the stylet (distal) and engages the second lumen of the endotracheal tube and prevents rotation of the endotracheal tube on the stylet. By cancelling the rotation of the DLT 40 with the counter rotation of the Stylet 10, the DLT 40 tube is mounted in such a way that the natural curvature of the endotracheal tube plastic memory is reversed. The reversal of the curvature results in the bronchial tube lumen 44 portion of the DLT 40 being diverted from left to right in a left-sided DLT 40 and for the bronchial cuff 62 to be kept on the right with a right-sided DLT 40. The DLT 40 may assume dimensions commonly designated as French gauges (Fr). Adult size DLT 40 may be 35, 37, 39, and 41 Fr gauges. Pediatric size DLT 40 may be 26, 28, and 32 Fr gauges.

In alternate embodiments, the semi-rigid stylet 10 may be configured as a long rod having a tubular design with a curved distal end and a defined proximal end which may be constructed of a medically approved material of metal or plastic that meets ASTM and/or ISO requirements. The stylet 10 is long enough to be inserted the entire length of the DLT 40 and attached tube extensions 108 to the bronchial cuff region 62. An alternate embodiment of the stylet 10 would accommodate DLT 40 having sizes 6-10 and the 26, 28, 32, 35, 37, 39, and 41 Fr gauges. The securing bar 22 of the stylet 10 serves to stabilize and lock the stylet 10 in a position favorable for intubation. For example, the stylet 10 may be passed through the bronchial lumen 48 and when almost completely inserted the securing re-curved bar 22 may be inserted through the tracheal tube orifice 114 and kept from moving within the tracheal tube extension 112. Similarly, the stylet 10 may be passed through the tracheal lumen 44 and when almost completely inserted the securing re-curved bar 22 may be inserted through the bronchial tube orifice 110 and kept from moving within the bronchial tube extension 108. Yet other embodiments provide for the stylet 10 to be hollow to allow the passage of oxygen or air during the process of intubation.

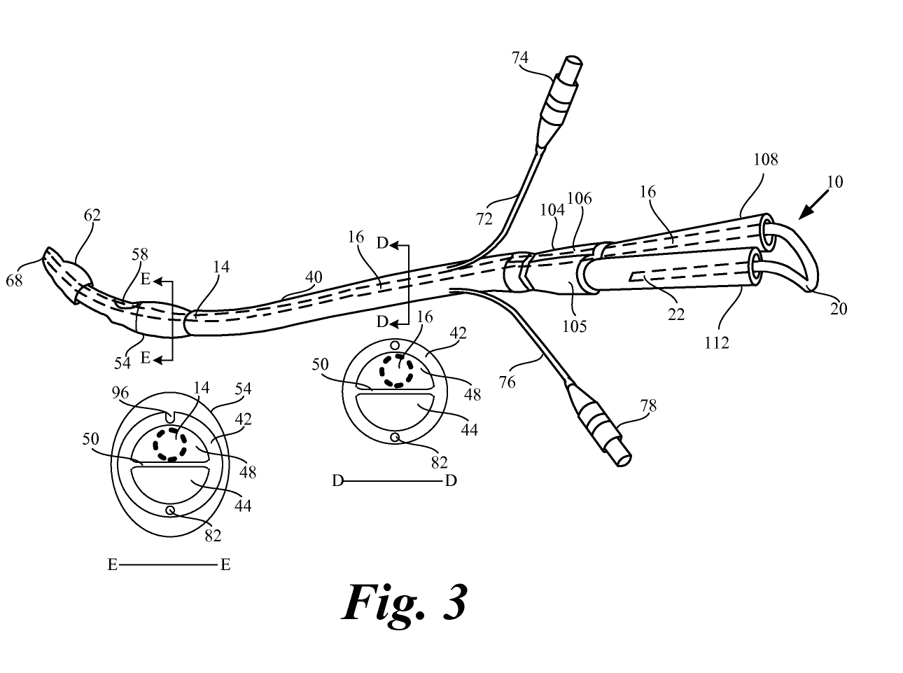

FIG. 3 schematically depicts the semi-rigid stylet 10 positioned in the bronchial channel 48 of a left-sided double lumen endotracheal tube 40. The securing re-curve bar 22 is shown parked or secured within the bronchial extension 108. Cross-section view D-D near the middle region of the left-sided DLT 40 shows the middle portion 16 of the stylet 10 occupying the tracheal lumen 44. Similarly, tracheal cuff 54 cross-section view E-E shows the distal portion 14 of the stylet 10 occupying the tracheal lumen 44.

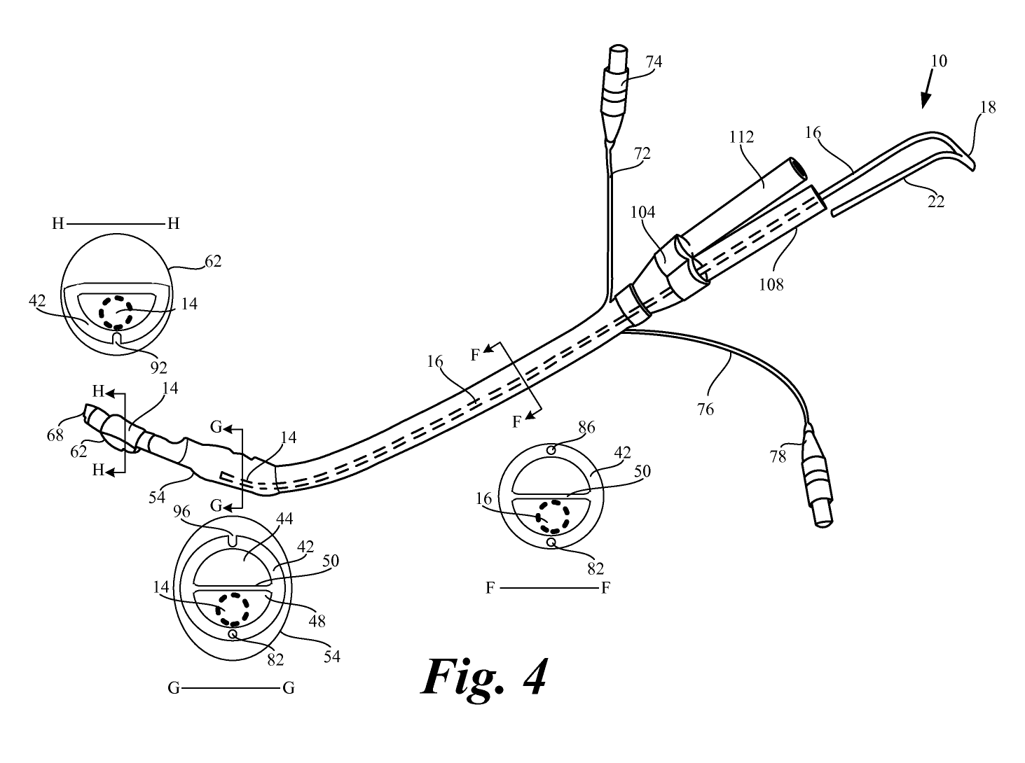

FIG. 4 depicts schematically depicts the semi-rigid stylet 10 positioned in and partially retracted from the bronchial channel 48 of a right-sided double lumen endotracheal tube 40 and bronchial tube extension 108. The securing re-curve bar 22 is shown free to clockwise rotate by an operator's thumb engaging with the stylet's 10 proximal handle 18 to clockwise spiral the DLT 40 on insertion into the patient. Cross-section view E-E near the middle region of the DLT 40 shows the middle portion 16 of the stylet 10 occupying the bronchial lumen 48. The tracheal cuff 54 cross-section view G-G shows the curved distal portion 14 of the stylet 10 occupying the bronchial lumen 48. Similarly, the bronchial cuff 62 cross-section view H-H shows the curved distal portion 14 of the stylet 10 occupying the bronchial lumen 48.

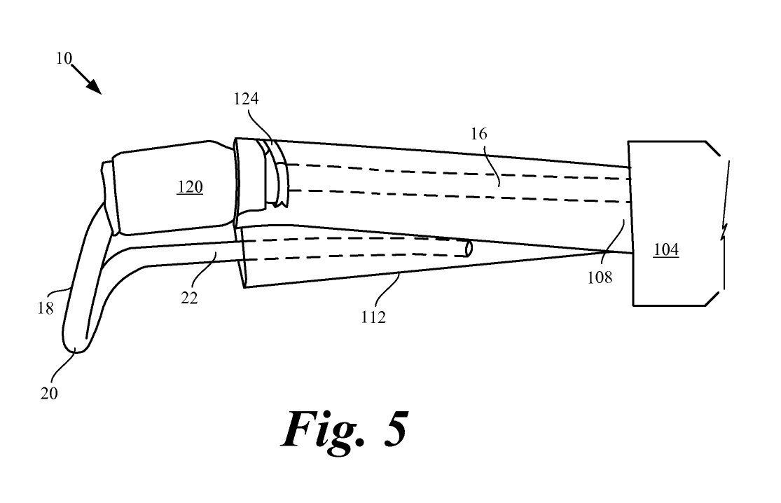

FIG. 5 schematically depicts an alternate embodiment of the semi-rigid stylet 10 fitted with an adjustable plug 120 engaged with the bronchial tube extender 108. The adjustable plug 120 slides back and forth to impart finer tuning or gradual angulations as needed for placement of the bronchial orifice 68. Friction pad 124 secures against the internal surfaces bronchial tube extension 108. DLT 40 advancement and/or back and forth movement may be achieved by an inward-to-outward displacement action that can be achieved by direct pull-and-push action or rotational or counter-rotational (twisting) action applied to the adjustable plug 120. The adjustable block 120 serves as a stylet adjustment aid in that the sliding block 120 engages the double lumen tube proximally to allow some adjustment for length variations between double lumen tubes 40.

Other embodiment for the stylet 10 herein described provide for the distal portion curved with a radius that produces a 45-90 degree curvature in the distal 25% of the length and the design may carry this radius to the 50% point or to the point that 180 degree curve may be achieved. The usual curvature would produce a 60-90 degree in change of direction. Other embodiments of the stylet include a curved portion at the proximal end or an added part that provides a surface or appendage for placing the thumb for partial extraction of the device from the DLT 40 and allowing the tube to rotate clockwise during this maneuver. Yet other embodiments of the stylet provide for an extension of the rod or tube at the proximal end that curves back in a direction toward the distal tip of the device and allows the operator to place the rod into the second tracheal lumen to stabilize the DLT 40 and prevent rotation of the DLT 40 during insertion into the mouth and into the glottic opening. The re-curved portion 22 keeps a left-sided DLT stable on a way that is counter to the natural curve of the DLT 40 plastic curve but on the right-sided tube keeps the tube positioned in plane with the natural plastic curve. Both the right and left-sided DLTs 40 result with the bronchial balloon or cuff 62 properly aimed toward the appropriate side of the lung. The semi-rigid stylet 10 design provides for a method to load the stylet 10 into the bronchial lumen 48 of the DLT and loading the re-curved proximal portion 22 into the tracheal lumen extension 112 so that when the tube is engaged into the glottic opening the stylet 10 may be removed partially until the tracheal lumen is free and the DLT 40 may be rotated to clockwise direction as it is inserted into the trachea under direct vision from a GlideScope video laryngoscope. The adjustable plug 120 device may be used when a shorter DLT 40 is used.

Method for intubation advantageously employ the properties of the stylet's 10 distal curvature 14, thumb manipulation of the proximal region 20, and semi-rigidity to permit accurate placement of the bronchial cuff 62 of either a left or right-sided DLT 40 to the appropriate lung location. The elongated rigid stylet 10 provides more rigidity to the stylet 10 while advancing the DLT 40. The stylet 10 modified distal curvature 14 may be suitable for DTL sizes of 6-10. The proximal end 18 has a recurved feature or securing bar 22 that is designed to be used to stabilize and lock the DLT 40 in a position favorable for intubation. The Stylet 10 may be passed into the bronchial lumen extension 108 and then when almost completely inserted the re-curved portion or securing bar 22 may be inserted into the tracheal tube extension 112 of the DLT 40. The intubation may be then carried out by advancing the DLT 40 to engage the entrance to the glottis and the stylet 10 may be withdrawn from the tracheal side and as the DLT 40 is advanced. The DLT 40 may be rotated clockwise to prevent the bronchial portion from engaging the vocal cord and then preventing the tracheal orifice 58 from engaging the arytenoids. The extraction of the stylet 10 may be then carried out and the intubation may be completed with the bronchial cuff 62 of the bronchial lumen 48 on the appropriate side of the lung.

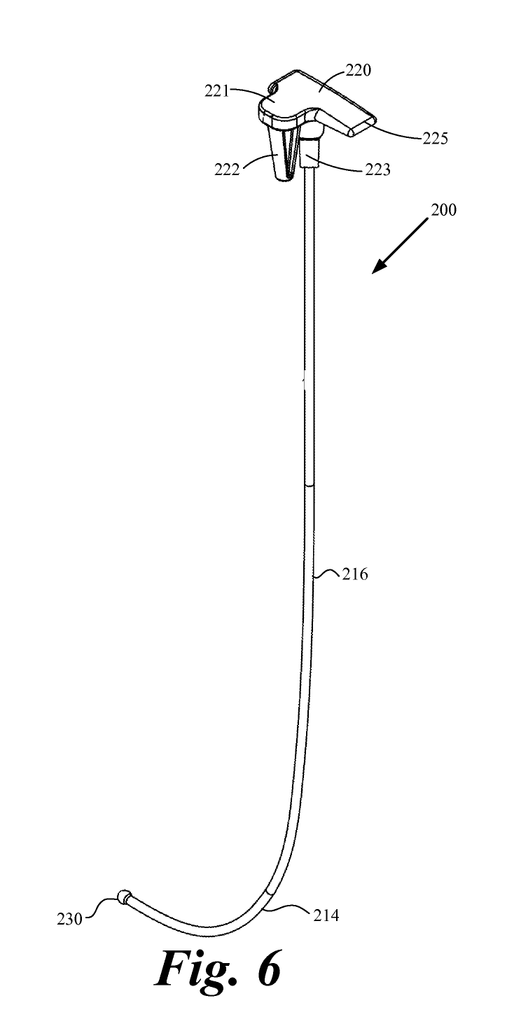

FIG. 6 is a perspective view that schematically depicts an alternate embodiment of a stylet 200 fitted with a handle grasp. The stylet 200 can accommodate left and right-handed DLT 40 having sizes 6-10 and the 26, 28, 32, 35, 37, 39, and 41 Fr gauges. The stylet 200 includes a substantially curved shaft portion comprising a distal region 214 and a substantially linear region 216. The linear region 216 connects with a handle 220. The handle 220 has a flat extension 221 that holds a V-shape lock projection 222 and a thumb extension 225 that provides a surface to engage a user's thumb to assist in steering the placement or routing of the DLT 40's bronchial cuff 62. The handle 220 may be connected to the linear region 216 via a handle base 223. The distal curved region 214 terminates with a ball 230 that is preferably forged with the curved region 214. Materials used in manufacturing the distal curve region 214, the linear region 216, and the ball 230 meet ASTM and/or ISO requirements and may include grade 316 stainless steel. The stylet 200's curved and linear regions 214 and 216 may have an approximate 3.2 mm diameter. The ball 230 has a smooth finish and may have a 4.8 mm diameter. The ball 230 serves to extend beyond the DLT 40's bronchial orifice 68 to guide the distal placement of the bronchial cuff 62 without causing tissue damage as the smooth surface of the ball 230 is not prone to piercing nearby tissue.

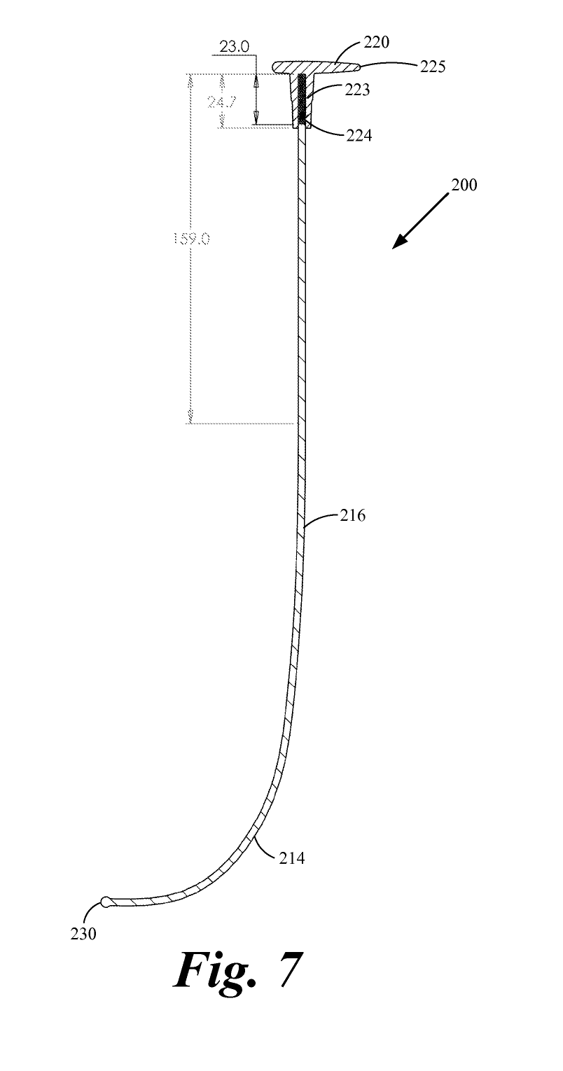

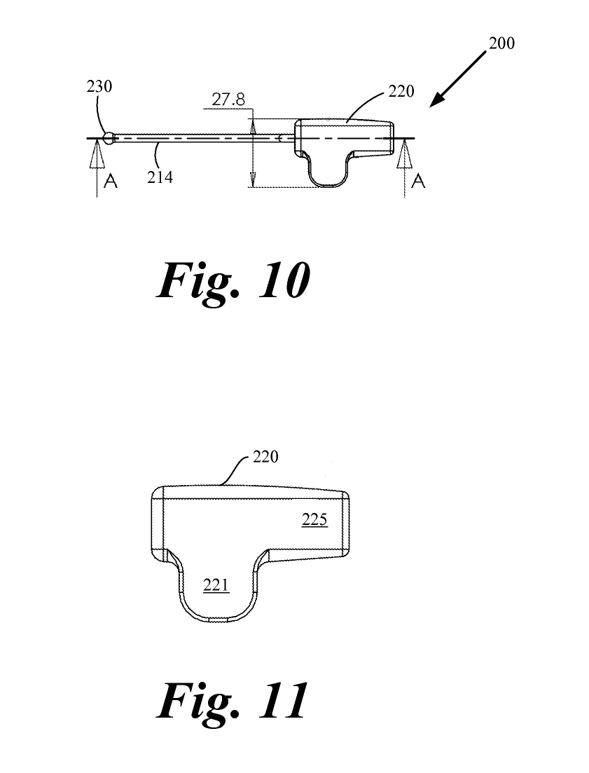

FIG. 7 depicts a side cross-sectional view of the stylet 200 of section A-A of FIG. 10 below. The stylet 200's curved and linear regions 214 and 216 shaft portions may be made from a distal section 234, a middle section 238, and a proximal section 242. The lengths of the distal section 234, the middle section 238, and the proximal section 242 may be varied to make the shaft portion of the stylet 200 assume different dimensions to accommodate a given patient's lung anatomy. The proximal section 242 has a knurled portion 224 that engages the handle base 223 to secure the handle 220 with the sytlet's proximal shaft section 242.

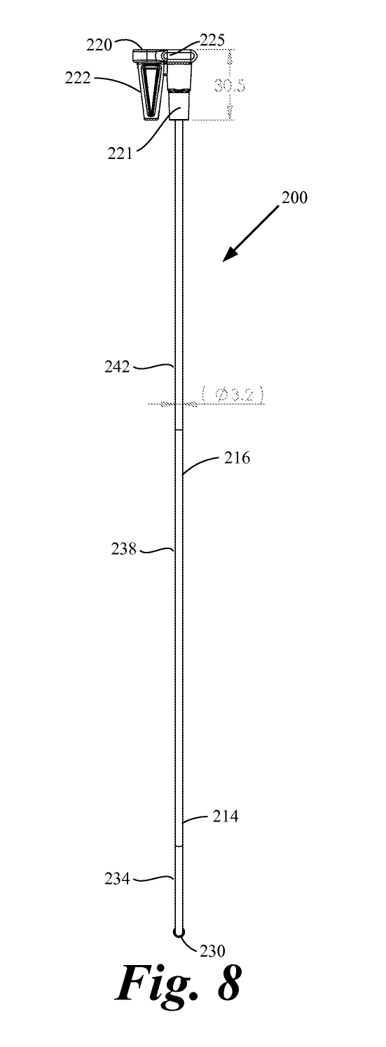

FIG. 8 depicts a rear view of the stylet 200. The length of the handle's 220 base 221 may be approximately 30.5 mm and is shown engaged with the proximal shaft portion 242. The proximal shaft 242, mid section 238, and distal curved section 234 has a diameter of approximately 3.2 mm.

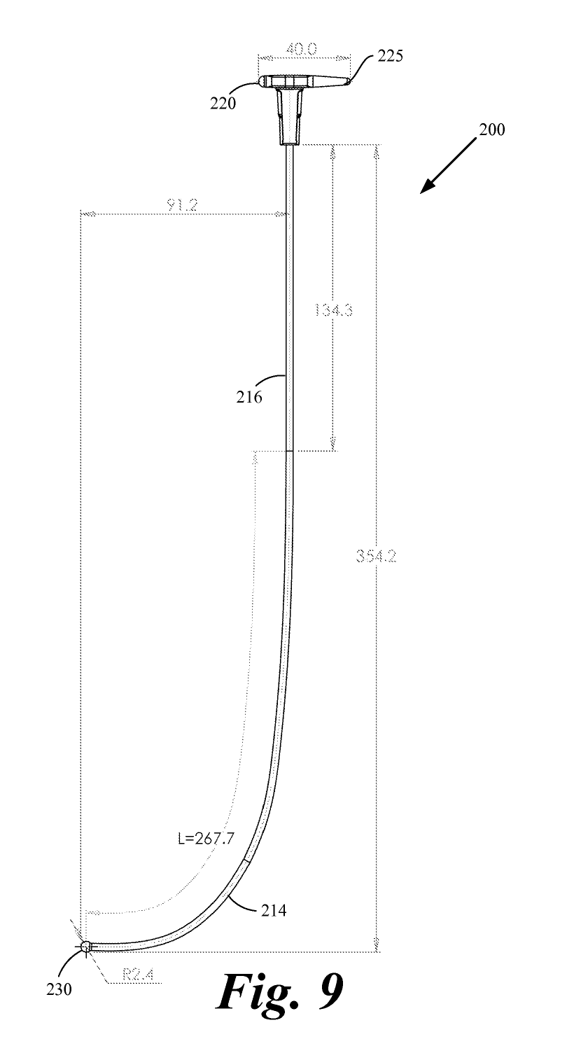

FIG. 9 depicts a side view of the stylet 200. The length of from the midpoint of the ball 230, the distal curved section 234, and the middle section 238 may be approximately 268 mm. The proximal section 342 may be approximately 134 mm. The length from the bottom of the base 223 and ball 230 may be approximately 354 mm.

FIG. 10 depicts a top view cross-sectional view along section A-A of the stylet 200. The handle width from the end of the extension 221 to the edge of the handle 220 may be approximately 28 mm.

FIG. 11 depicts top view of the stylet handle 222 and illustrates the extension 221 and the thumb region 225 that may be located approximately at a right angle to the extension 221.

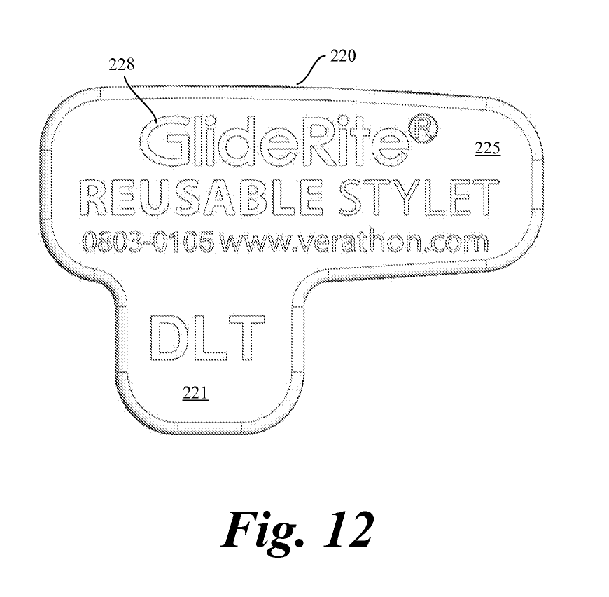

FIG. 12 depicts top view of the stylet handle 222 illustrating alphanumeric information 228 formed thereon. The alphanumeric information 228 reads “GlideRite® Reusable Stylet 0803-.www.verathon.com DLT”. The term “DLT” resides in the extension 221 from which locking V-shaped tab 222 extends from. Other information may be substituted for the alphanumeric information 228.

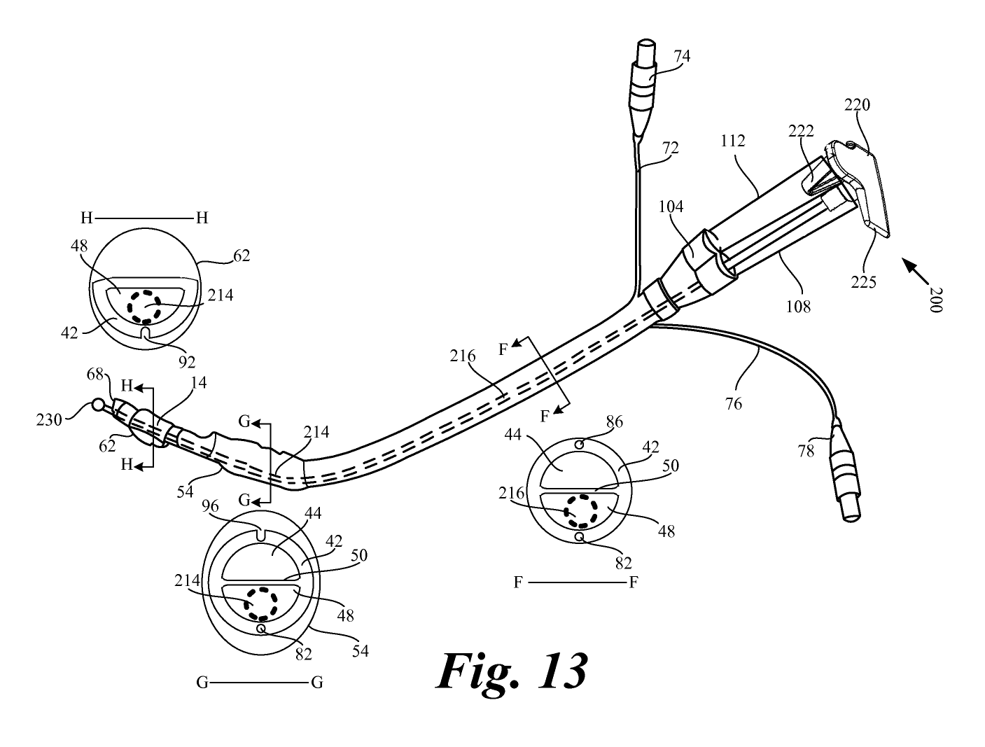

FIG. 13 schematically depicts the semi-rigid stylet 200 positioned in the bronchial channel of a right-sided double lumen endotracheal tube to position the ball 230 beyond the bronchial lumen orifice 68. The semi-rigid stylet 200 may be positioned in a right sided DLT-40 in which the locking V-shaped tab 222 may be inserted into tracheal extension tube 112. The shaft portions 242, 238, and 234 are projected through the bronchial lumen 48 to place the distal curved region 214 through the bronchial cuff 62 region and so that ball 230 projected beyond bronchial orifice 68. The ball 230 may then be routed by thumb action manipulation of the thumb engagement section 225 of handle 220 to steer and place the bronchial cuff 62 at a desired location of the patient's lung. The locking V-tab 222 motion may be restrained within the tracheal tube extension 112 to allow more controlled movement of the ball 230.

While the preferred embodiment of the invention has been illustrated and described, as noted above, many changes can be made without departing from the spirit and scope of the invention. Accordingly, the scope of the invention is not limited by the disclosure of the preferred embodiment. Instead, the invention should be determined entirely by reference to the claims that follow.

This provisional patent application is intended to describe one or more embodiments of the present invention. It is to be understood that the use of absolute terms, such as “must,” “will,” and the like, as well as specific quantities, is to be construed as being applicable to one or more of such embodiments, but not necessarily to all such embodiments. As such, embodiments of the invention may omit, or include a modification of, one or more features or functionalities described in the context of such absolute terms.

1. A stylet for engaging the bronchial lumen of a double lumen endotracheal tube comprising:
a distal curved region;
a proximal handle region; and
a linear region located between the distal curved and proximal handle regions,
wherein manipulation of the proximal handle region steers the distal portion of the bronchial lumen to a desired location in the lung of a patient.

2. The stylet of claim 1, wherein the distal portion is curved by approximately 25% the length of the stylet.

3. The stylet of claim 1, wherein the proximal handle region includes a projection for engaging a user's thumb.

4. The stylet of claim 1, wherein the proximal handle region includes an adjustable plug configured to engage with a bronchial extension of the double lumen endotracheal tube.

5. The stylet of claim 1, wherein the proximal handle region includes a frictional pad configured to engage with a tracheal extension of the double lumen endotracheal tube.
